(19) United States
(12) Patent Application Publication
(10) Pub. No.: US 2002/0120948 A1
(43) Pub. Date: Aug. 29, 2002

(54) Methods for expressing gene products

(76) Inventor: Amanda Fisher, London (GB)

Correspondence Address: PALMER & DODGE, LLP, KATHLEEN M. WILLIAMS, 111 HUNTINGTON AVENUE, BOSTON, MA, 02199, US

(21) Appl. No.: 09/957,674

(22) Filed: Sep. 20, 2001

Related U.S. Application Data
(63) Continuation-in-part of application No. PCT/GB00/01225, filed on Mar. 30, 2000.

(30) Foreign Application Priority Data
Mar. 30, 1999 (GB) GB 99073660

(51) Int. Cl. A61K 48/00; G01N 33/00; A61K 38/19

(52) U.S. Cl. 800/3; 424/93.21; 424/85.1

(57) ABSTRACT

The invention provides a method for modulating the amount of a transgene product in a host organism. In one aspect, the invention is used to provide large quantities of a desired transgene product in a transgenic animal, which can then be isolated to produce pharmaceutical compositions in which the transgene product is the active ingredient. In one aspect, the method comprises the steps of providing a host organism comprising a regulatable population of cells which express a transgene encoding the transgene product and administering an agent that regulates the number of transformed cells, allowing the level of the transgene to be modulated. The regulatable population of cells can be transformed with the transgene and introduced into the host organism or the regulatable population of cells comprising the transgene can be generated by germline transformation of a transgenic animal after transformation of one or more germline cells with the transgene.

### RELATED APPLICATIONS

This application claims priority under 35 U.S.C. § 120 to PCT/GB00/01225, filed Mar. 30, 2000 and also claims priority under 35 U.S.C. § 119(a)-(d) to GB 990736.0 filed Mar. 30, 1999.

### FIELD OF THE INVENTION

The invention relates to a method for expressing heterologous nucleic acids encoding transgene product(s) of interest in a host organism.

### BACKGROUND OF THE INVENTION

The regulation of transgenes within a host organism is a desirable goal to which a variety of solutions have been proposed. For example, transgenes may be placed under the control of inducible transcriptional control elements and the transcription may be induced either in response to a biological stimulus, such as hypoxia, or in response to administration of a secondary agent, such as a small molecule. In certain cases, the transgene may be regulated in response to a disease state, such as infection, for example, by placing it under the control of sequences responsive to cytokines naturally expressed in response to infection. Alternatively, the transgene may be placed under the control of sequences that render it susceptible to tissue-specific regulation, such that the transgene is active only in certain tissue types; and/or the transgene may be developmentally regulated, such that its expression is activated only at certain stages of development.

In each of the foregoing methods, transgene expression is controlled by upregulating (or downregulating) gene expression in response to defined stimuli. Certain other techniques exploit modulation of gene copy number in the host in order to achieve similar effects. For example, where the transgene is encoded within a viral genome, virus replication in the host may be induced by appropriate techniques.

### SUMMARY OF THE INVENTION

A new method for regulating transgene expression is disclosed. The method, according to the invention, is unlike the methods of the prior art since it is not dependent on manipulation of transcriptional control elements. In the present invention, gene expression is modulated by regulation of cell proliferation. Therefore, it is the number of cells in the host organism that comprise the transgene, as opposed to the activity of the transgene itself, which affects the total amount of transgene product. In one embodiment, the invention relates to regulated transgene expression in lymphocytes by selective increase in the number of lymphocytes upon exposure to an antigen.

In one aspect, the method comprises the steps of providing a host organism comprising a regulatable population of cells at least a portion of which expresses a transgene encoding the transgene product and administering an agent that regulates the number of transformed cells, thus permitting the level of the transgene to be modulated.

In another aspect, the method for modulating the amount of a transgene product produced in a host organism comprises the steps of: i) transforming a regulatable population of cells with a transgene, ii) introducing the population of transformed cells into the host, thus, allowing for production of the transgene product, and iii) administering an agent that regulates the number of the transformed cells, thereby modulating the amount of transgene product produced in the host organism. In one embodiment, the agent used causes the number of cells to increase whereas in another embodiment the agent causes a decrease in cell number. The regulatable population of cells can also be a clonal population of cells, such as red blood cells, including lymphocytes. The host organism in which the transgene product is expressed is preferably a mammal and the agent used to modulate cell number may be a cytokine or, in the case of immune cells, such as lymphocytes, may be an antigen.

In a further aspect, the regulatable population of cells comprising the transgene is generated through germline transformation of a host organism thereby generating a transgenic host organism comprising regulatable cells. Modulation of the transgene product is achieved by administering the agent to the transgenic host organism. In still a further aspect, regulatable cells comprising the transgene are isolated from the host organism and introduced into a second host organism (which may or may not be transgenic) and the agent is administered to the second host organism.

A second aspect of the invention takes advantage of clonal expansion of lymphocytes in response to antigen presentation to induce selective proliferation of transformed populations of lymphocytes. According to this aspect of the invention the amount of transgene product in a host organism is controlled by: i) immunizing the host with an antigen, ii) isolating lymphocytes from the host, iii) transforming the lymphocytes with a transgene that encodes the transgene product, iv) reintroducing the lymphocytes to the host, and v) administering a booster immunization of the antigen to the host, in order to modulate clonal expansion of the transformed lymphocytes. In one aspect, the method uses a primary immunization and one or more booster immunizations. In another aspect of the invention, the lymphocytes are expanded in vitro prior to reintroduction into the host. In addition, the isolated cell population that contains lymphocytes can be further enriched for lymphocytes prior to transformation or reintroduction into the host.

The transgene product produced may be a polypeptide, such as an enzyme, a transcription factor, a growth factor, a hormone, a toxin, an antibody, a clotting factor, apolipoprotein Al, α-1 antitrypsin, or a peptide drug. In addition, the polypeptide may be a clotting factor, for example, Factor VIII or Factor IX. Polypeptide transgene products may also be derived from viral sequences. The transgene product described in this invention is not limited to a polypeptide and may be a nucleic acid. The nucleic acid may be DNA (e.g., such as a viral sequence) or RNA, including antisense RNA and ribozymes.

### BRIEF DESCRIPTION OF THE FIGURES

The objects and features of the invention can be better understood with reference to the following detailed description and accompanying drawings.

FIG. 1 is a schematic diagram showing stages in an immuno-regulated gene therapy protocol according to one aspect of the invention.

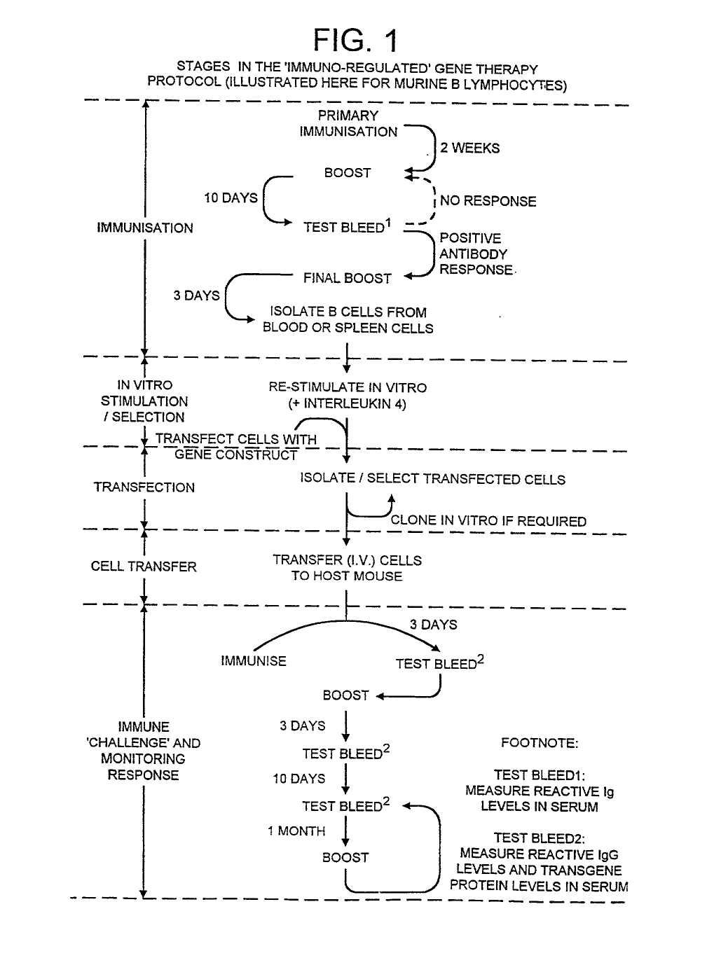

FIG. 2 shows the results obtained in mice immunized with PE alone (no cells), mice given cells with PE and mice given cells alone.

### DETAILED DESCRIPTION

The invention provides a novel method for regulating the amount of a transtransgene product that is produced in a host organism. Unlike the methods found in the prior art, the amount of transgene product produced from a transgene is regulated by modulation of the number of cells that comprise the transgene. Cell number is modulated by exposure to an agent that either induces cell proliferation or cell death.

### Definitions

The following definitions are provided for specific terms that are used in the following written description.

As defined herein a “population of cells” refers to a plurality (at least two cells, preferably, at least 100, more preferably, at least 1000, still more preferably, at least 10⁵, 10⁶, 10⁷, 10⁸ cells) which are substantially all of the same type (e.g., comprising at least about 95%, at least about 98%, to 100% of cells of the same type, i.e., cells which comprise substantially identical genomic sequence (having 99%-100% identity) in the population and which express substantially the same transcripts and polypeptides (95%, and preferably 99%-100% of transcripts, polypeptides in any given cell are the same as the transcripts/polypeptides in a cell which is “substantially the same”).

As used herein, a “regulatable population of cells” is any population of cells in which the total cell number of the population can be selectively regulated, i.e., the total number of cells may be increased or decreased in response to administration of an agent. Advantageously, the cells will be part of a tissue or organ capable of controlled proliferation, such as blood cells. Other regulatable cells include, but are not limited to, apoptotic cells, hematopoietic stem cells, antigen presenting cells (e.g, macrophages, monocytes, dendritic cell, a macrophage, a B cell, a mast cell, a parenchymal cell, a kupffer cell, or a fibroblast cell.), T cells, monocytes, basophils, natural killer cells, erthrocytes, megakaryocytes, platelets, basophils, endothelial cells, leukocytes, fibroblasts, endothelial cells, chondrocytes, osteoblasts, and their precusor cells. fibroblasts, eosinophils, keratinocytes, astrocytes, microglial cells, thymic cortical epithelial cells, Schwann cells, retinal pigment epithelial cells, myoblasts, hepatocytes, vascular smooth muscle cells, enterocytes, thyrocytes and kidney tubule cells. Cells may be of epithelial, connective, muscle tissue, or of nervous tissue origin, so long as these are capable of modulating their numbers in response to an agent. The selective nature of the regulation process according to the invention ensures that cells, which are subject to a regulation of proliferation in response to administration of the agent, increase or decrease upon exposure to the agent. Preferably, the cells are B and/or T lymphocytes; advantageously, they are memory B and/or T lymphocytes, that is lymphocytes that naturally persist for an extended period of time in an organism in order to preserve immune recognition memory. As used herein, an “increase” in cell numbers refers to at least a doubling of cell numbers, while a “decrease” in cell numbers refers to at least a two-fold decrease in cell numbers. A population of “regulatable cells” may comprise some numbers of non-regulatable cells, for example, 20% or less, 10% or less, 5% or less, and preferably, 2% or 1% or less, of non-regulatable cells.

As used herein, a “transformed population of regulatable cells” comprises cells, a plurality of which comprise a transgene which can express the transgene product at least under conditions in which the regulatable cells are regulated (e.g., at least when the regulatable cells are exposed to the agent). However, the transcription and translation of the transgene are not controlled by the agent. The agent acts to increase or decrease the levels of the transgene product by increasing or decreasing the number of cells comprising the transgene. Preferably, at least 1%, at least 5%, at least 10%, at least 20%, at least 30%, at least 40%, at least 50%, or at least 60%-100% of the regulatable cells within a transformed population of regulatable cells comprise the transgene.

As used herein, “regulation of proliferation” refers to modification of the number of cells present in response to administration of an agent. Preferably, proliferation is selective, in that only the regulatable population of cells of which a proportion was transformed with the transgene proliferates in response to the agent. The regulation of proliferation of cells by the method of the invention results in the modulation in the levels of production of the transgene product encoded by the transgene, meaning that an altered quantity of transgene product will be produced in the host organism.

As used herein, “modulating the amount of transgene product encoded by the transgene” means any increase or decrease in the total amount of transgene product encoded by the transgene. In one aspect, modulating means an increase or decrease of at least 1% of the total transgene product produced. In another aspect, the increase or decrease in the total transgene product produced is at least 5%-25%, or at least 25% to 50%. In addition, the change in transgene product may be at least 2-, 4-, 5-, 10-, 20-, 25- or 50 fold. The amount of transgene product increases or decreases because the number of transformed cells producing the transgene product changes; however, in some aspects the amount of transgene product produced by each transformed cell may also change.

As used herein, “transgene” means a nucleic acid sequence encoding a transgene product which is partly or entirely heterologous, i.e., foreign, to a cell into which it is introduced or, is homologous to an endogenous gene of cell into which it is introduced, but which is designed to be inserted, or is inserted, into the genome of the cell in such a way as to alter the genome of the cell into which it is inserted (e.g., it is inserted at a location which differs from that of the natural gene or its insertion results in a knockout or disruption of an endogenous gene). A transgene can include one or more transcriptional regulatory sequences and other nucleic acid sequences, such as introns, that may be necessary for optimal expression of a selected nucleic acid. Thus, transcriptional and translational control of the transgene to produce the transgene product of interest may be achieved by any of a variety of suitable techniques known to those skilled in the art. In a preferred aspect of the invention, the level of expression of a transgene, in addition to being controlled through regulation of the number of cells comprising the transgene, may be subject to control at the transcriptional or translational level, thus introducing a further level of regulation into the system.

As used herein, “transforming” refers to transfer of genetic information into a cell. Thus, the term includes viral transduction, transfection, electroporation, protoplast fusion, and nucleic acid delivery by any other means. The term “transforming” also includes modification or deletion of any endogenous nucleic acid using methods known in the art, for example the use of gene “knockout” technology to delete all or part of a gene, or the use of recombinant technology to selectively insert DNA sequences. The modification can modulate the levels of transgene product produced.

As used herein, “transgene product” refers to a polypeptide or nucleic acid molecule, which may possess regulatory function, catalytic function or any other function that is desirable within a host organism. Thus, the transgene product may be: an enzyme, a transcription factor, a growth factor or a hormone, a toxin, an antibody, a clotting factor such as Factor VIII or Factor IX, Apolipoprotein Al, α-1 antitrypsin, a peptide therapeutic agent; or a viral polypeptide. In addition, the transgene product may be a ribozyme, or a sense or antisense RNA molecule. The RNA molecule can be comprised of natural ribonucleotides or modified ribonucleotides, which are preparable according to procedures known in the art, and may contain mixtures thereof. In addition, the nucleic acid molecule may be derived from a viral genome.

As used herein, an “agent” is a molecule able to cause an increase or decrease in the number of cells by either stimulating an increase in cell proliferation or by inducing cell death. For example, selected cytokines induce lymphocyte (F-cell or B cell) proliferation, or proliferation of other blood cells such as macrophages, neutrophils or other leukocytes, mast cells and antigen-presenting cells. In addition, agents such as bclxs, myc, p53, bax, bak, ced transgene products, Fas ligand (FasL), Fas receptor, lymphotoxin, CD40L, and TNF- alpha can be used to reduce cell numbers, e.g., by inducing apoptosis. Other agents include, but are not limited to, antigens that can elicit antigen-specific T cell and B cell lymphocyte responses thereby increasing the number of immune response cells. Growth factors or hormones which act on any of the above regulatable cell types are encompassed within the scope of the invention, including, but not limited to: Stem Cell Factor (SCF; also known as mast cell growth factor, or Steel Factor), TGF-alpha, TGF-beta, VEGF, macrophage colony stimulating factor (M-CSF), keratinocyte growth factor (KGF), heregulin (HRG), insulin, insulin-like growth factors I and II (IGF-I and IGF-II), epidermal growth factor (EGF), interleukins (e.g., IL-8), macrophage colony-stimulating factor (M-CSF), erythropoietin (EPO), platelet-derived growth factor (PDGF), fibroblast growth factor (FGF), transforming growth factors alpha and beta (TGF- [alpha] and TGF- [beta]), hepatocyte growth factor (HGF), and nerve growth factor (NGF). Agents are not limited to polypeptides and may be chemical substances or biological organisms, such as an infecting organism or a virus that has been optionally engineered to express a molecule/agent that modifies cell number or a virus/infecting organism which itself may be cytolytic or transforming.

As used herein, “organism” is any desired multi-cellular organism. The method of the invention involves the regulation of the proliferation of cells transformed with a transgene encoding a transgene product within the organism in question. Advantageously, the organism possesses an immune system, which allows a preferred embodiment of the invention to be practiced, where clonal expansion of transformed lymphocytes is induced by antigen presentation. Preferably, the host organism refers to a member of the subphylum Chordata. It is intended that the term encompass any member of this subphylum, including, but not limited to humans and other primates, rodents (e.g., mice, rats, and guinea pigs), lagamorphs (e.g., rabbits), bovines (e.g, cattle), ovines (e.g., sheep), caprines (e.g., goats), porcines (e.g., swine), equines (e.g., horses), canines (e.g., dogs), felines (e.g., cats), domestic fowl (e.g., chickens, turkeys, ducks, geese, other gallinaceous birds, etc.), as well as feral or wild animals, including, but not limited to, such animals as ungulates (e.g., deer), bear, fish, lagamorphs, rodents, birds, etc. It is not intended that the term be limited to a particular age or sex. Thus, adult and newborn subjects, as well as fetuses, whether male or female, are encompassed by the term.

As used herein, “immunization” refers to the administration of antigen in order to induce the proliferation of antigen-specific lymphocytes in the organism (e.g., an “immune response”). As used herein, “a primary immunization” refers to the first time an antigen is administered to the host organsim. The term “booster immunizations” refer to additional administration of the antigen to the host organism.

As used herein, “agent administered” means to introduce the agent (e.g., such as by injection, ingestion, inhalation, and/or topical application) into the host organism at a concentration such that it modulates cell numbers of the regulatable population of cells. The term “administration” also encompasses production of an agent in vivo in the organism by natural or engineered biological means, in response to natural or artificial stimuli (e.g., the agent may be the transgene product of an inducible transgene).

As used herein, a “clonal population” refers to a population of cells derived from a single progenitor cell by cell division.

As used herein, “regulation of clonal expansion” includes increasing, decreasing, stabilizing or reversing the rate of clonal expansion. Thus, a clonal lymphocyte population may be induced to expand or contract in response to booster immunizations. As used herein, “regulation” of clonal expansion must be accompanied by an at least two-fold expansion or contraction of cell numbers or an at least 5% increase or decrease in cell numbers (and preferably, an at least 10%, at least 20%, at least 30%, at least 40%-100% increase or decrease). Contraction (decreases in cell numbers) may in particular be induced by immunization with peptides designed to induce contraction of lymphocyte clonal populations. Such peptides are, for example, altered peptide ligands (“APL”), which are modified forms of proliferation-inducing MFIC ligand peptides. Administration of APL is believed to induce anergy and possibly cell death in lymphocyte populations, both in T cells (Jameson, (1998) PNAS 95:14001-14002) and in B cells (Kouskoff et al., (1998) J. Exp. Med. 188:1453-64).

### Modulating Levels of a Transgene Product

The invention provides a method for modulating the amount of a transgene product in a host organism. In one aspect, the invention is used to provide large quantities of a desired transgene product in a transgenic animal, which can then be isolated to produce pharmaceutical compositions in which the transgene product is the active ingredient. However, the invention can also be use to modulate the amount of a transgene product in a host organism (e.g., such as a human being) in which the product has a therapeutic effect, thereby to regulate the efficacy and duration of the therapeutic effect by administering the agent to the host organism.

In one aspect, the method comprises the steps of providing a host organism comprising a regulatable population of cells which express a transgene encoding the transgene product and administering an agent that regulates the number of transformed cells, allowing the level of the transgene to be modulated. The regulatable population of cells can be transformed with the transgene and introduced into the host organism or the regulatable population of cells comprising the transgene can be generated by germline transformation of a transgenic animal after transformation of one or more germline cells with the transgene.

### Vector Construction

In general, a transgene according to the present invention will comprise an expressed nucleotide sequence, which may be transcribed to RNA and optionally translated to produce a polypeptide, and a vector.

A vector is a tool that allows or facilitates the transfer of an entity from one environment to another. By way of example, some vectors used in recombinant DNA techniques allow entities, such as a segment of DNA (i.e. a heterologous cDNA segment), to be introduced into a target cell. Once within the target cell, the vector may act as a unit of DNA that replicates, that remains episomal, or that integrates into the host genome. Examples of vectors used in recombinant DNA techniques include plasmids, chromosomes, artificial chromosomes or viruses.

Non-viral delivery systems include but are not limited to DNA transfection methods. Here, transfection includes a process using a non-viral vector to deliver a gene to a target mammalian cell.

Typical transfection methods include electroporation, DNA biolistics, lipid-mediated transfection, compacted DNA-mediated transfection, liposomes, immunoliposomes, lipofectin, cationic agent-mediated, cationic facial amphiphiles (CFAs) (Nature Biotechnology (1996) 14; 556), and combinations thereof.

As used herein, “plasmid” refers to discrete elements that are used to introduce heterologous DNA into cells for either expression or replication thereof Selection and use of such vehicles are well within the skill of the artisan. Many plasmid vectors are available, and selection of appropriate vector will depend on the intended use of the vector, i.e. whether it is to be used for DNA amplification or for DNA expression, the size of the DNA to be inserted into the vector, and the host cell to be transformed with the vector. Each vector contains various components depending on the host cell for which it is compatible. The plasmid vector components generally include, but are not limited to, one or more of the following: an origin of replication, one or more marker genes, an enhancer element, a promoter, a transcription termination sequence, a polyadenylation signal, intronic sequences, a signal sequence and any other sequences necessary to regulate transcription and/or translation. Such regulatory elements are described, for example, in Goeddel; 1990, Gene Expression Technology: Methods in Enzymology 185, Academic Press, San Diego, Calif.

The term “promoter” is used in the normal sense of the art, e.g. sequences which enable RNA polymerase binding and transcription initiation in the Jacob-Monod theory of gene expression.

The term “enhancer” refers to a DNA sequence which is not necessarily involved directly in transcription initiation, but is capable of enhancing transcription. The positioning of enhancers relative to the promoter is flexible, and enhancers are active in an orientation-independent manner. Enhancers bind to additional components which may interact with the transcription initiation complex and thus upregulate transcription.

Plasmid vectors also generally contain nucleic acid sequences that enable the vector to replicate in one or more selected host cells. Typically in cloning vectors, this sequence is one that enables the vector to replicate independently of the host chromosomal DNA, and includes origins of replication or autonomously replicating sequences. Such sequences are well known for a variety of mammalian cells, bacteria, yeast and viruses. The origin of replication from the plasmid pBR322 is suitable for most Gram-negative bacteria, the 2μ plasmid origin is suitable for yeast, and various viral origins (e.g. SV 40, polyoma, adenovirus) are useful for cloning vectors in mammalian cells. Generally, the origin of replication component is not needed for mammalian expression vectors unless these are used in mammalian cells competent for high level DNA replication, such as COS cells.

Most expression vectors are shuttle vectors, i.e., they are capable of replication in at least one class of organisms but can be transfected into another class of organisms for expression. For example, a vector is cloned in E. coli and then the same vector is transfected into cells of the host organism even though it is not capable of replicating independently of the host cell chromosome. DNA may also be replicated by insertion into the host genome. DNA can be amplified by PCR and be directly transfected into the host cells without any replication component.

Advantageously, an expression (and cloning) vector may contain a selection gene also referred to as selectable marker. This gene encodes a protein necessary for the survival or growth of transformed host cells grown in a selective culture medium. Host cells not transformed with the vector containing the selection gene will not survive in the culture medium. Typical selection genes encode proteins that confer resistance to antibiotics and other toxins, e.g., ampicillin, neomycin, methotrexate or tetracycline, complement auxotrophic deficiencies, or supply critical nutrients not available from complex media.

The following markers, for example, have been used successfully in, inter alia, retroviral vectors. The bacterial neomycin and hygromycin phosphotransferase genes which confer resistance to G418 and hygromycin respectively (Palmer et al., (1987) PNAS 84:1055-1059; Yang et al., (1987) Mol. Cell. Biol. 7:3923-3928); a mutant mouse dihydrofolate reductase gene (dhfr) which confers resistance to methotrexate (Miller et al., (1985) Mol. Cell. Biol. 5:43 1-437); the bacterial gpt gene which allows cells to grow in medium containing mycophenolic acid, xanthine and aminopterin (Mann et al., (1983) Cell 33:153-159); the bacterial hisD gene which allows cells to grow in medium without histidine but containing histidinol (Danos and Mulligan, (1988) Proc. Natl. Acad. Sci. 85:6460-6464); the multidrug resistance gene (mdr) which confers resistance to a variety of drugs (Guild et al., (1988) Proc. Natl. Acad. Sci. 85:1595-1599; Pastan et al., (1988) Proc. Natl. Acad. Sci. 85:4486-4490) and the bacterial genes which confer resistance to puromycin or phleomycin (Morgenstern and Land, (1990) Nucleic Acid Res. 18:3587-3596).

All of these markers are dominant selectable markers and allow chemical selection of most cells expressing these genes. β-galactosidase can also be considered a dominant marker; cells expressing β-galactosidase can be selected by using fluorescence-activated cell sorting (FACS). In fact, any cell surface protein can provide a selectable marker for cells not already making the protein. Cells expressing the protein can be selected by using the fluorescent antibody to the protein and a cell sorter. Other selectable markers that have been included in vectors include the hprt and HSV thymidine kinase which allows cells to grow in medium containing hypoxanthine, amethopterin and thymidine.

Since the replication of vectors is conveniently done in E. coli, an E. coli genetic marker and an E. coli origin of replication are advantageously included. These can be obtained from E. coli plasmids, such as pBR322, Bluescript© vector or a pUC plasmid, e.g. pUC18 or pUC19, which contain both E. coli replication origin and E. coli genetic marker conferring resistance to antibiotics, such as ampicillin.

Suitable selectable markers for mammalian cells are those that enable the identification of cells competent to take up a vector containing the transgene, such as dihydrofolate reductase (DHFR, methotrexate resistance), thymidine kinase, or genes conferring resistance to G418 or hygromycin. The mammalian cell transformants are placed under selection pressure which only those transformants which have taken up and are expressing the marker are uniquely adapted to survive. In the case of a DHFR or glutamine synthase (GS) marker, selection pressure can be imposed by culturing the transformants under conditions in which the pressure is progressively increased, thereby leading to amplification (at its chromosomal integration site) of both the selection gene and the linked transgene DNA. Amplification is the process by which genes in greater demand for the production of a protein critical for growth, together with closely associated genes which may encode a desired protein, are reiterated in tandem within the chromosomes of recombinant cells. Increased quantities of desired protein are usually synthesized from thus amplified DNA.

Expression and cloning vectors usually contain a promoter that is recognized by the host organism and is operably linked to the transgene. Such a promoter may be inducible or constitutive. The promoters are operably linked to the transgene by removing the promoter from the source DNA and inserting the isolated promoter sequence into the vector. Both the native promoter sequence usually associated with the transgene in nature, if applicable, and many heterologous promoters may be used to direct amplification and/or expression of the transgene. The term “operably linked” refers to a juxtaposition wherein the components described are in a relationship permitting them to function in their intended manner. A control sequence “operably linked” to a coding sequence is ligated—in such a way that expression of the coding sequence is achieved under conditions compatible with the control sequences.

Transgene transcription from vectors in mammalian hosts may be controlled by promoters derived from the genomes of viruses such as polyoma virus, adenovirus, fowlpox virus, bovine papilloma virus, avian sarcoma virus, cytomegalovirus (CMV), a retrovirus and Simian Virus 40 (SV40), from heterologous mammalian promoters such as the actin promoter or a very strong promoter, e.g., a ribosomal protein promoter, and from the promoter normally associated with the coding sequence of the transgene, provided such promoters are compatible with the host cell systems.

Transcription of the transgene by higher eukaryotes may be increased by inserting an enhancer sequence into the vector. Enhancers are relatively orientation and position independent. Many enhancer sequences are known from mammalian genes (e.g. elastase and globin). However, typically one will employ an enhancer from a eukaryotic cell virus. Examples include the SV40 enhancer on the late side of the replication origin (bp 100-270) and the CMV early promoter enhancer. The enhancer may be spliced into the vector at a position 5′ or 3′ to the transgene, but is preferably located at a site 5′ from the promoter.

Promoter and enhancer elements have been isolated from a variety of eukaryotic sources including genes in yeast, insect and mammalian cells and viruses (analogous control elements, i.e., promoters, are also found in prokaryotes). The selection of a particular promoter and enhancer depends on the cell type of the regulatable population of cells. Some eukaryotic promoters and enhancers have a broad range of cells in which they can activate and/or modulate transcription while others are functional only in a limited subset of cell types (see, e.g., Voss et al., (1986) Trends Biochem. Sci. 11:287 for a review). For example, the SV40 early gene enhancer is very active in a wide variety of cell types from many mammalian species and has been widely used for the expression of proteins in mammalian cells (Dijkema et al., (1985) EMBO J. 4:761). Two other examples of promoter/enhancer elements active in a broad range of mammalian cell types are those from the human elongation factor 1α gene (Uetsuki et al., (1989) J. Biol. Chem., 264:5791; Kim et al., (1990) Gene 91:217; and Mizushima et al., (1990) Nuc. Acids. Res., 18:5322) and the long terminal repeats of the Rous sarcoma virus (Gorman et al., (1982) PNAS 79:6777) and the human cytomegalovirus (Boshart et al., (1985) Cell 41:521). This list is not intended to be limiting and additional regulatory sequences can be obtained from the Eukaryotic Promoter Data Base (EPDB) as is known in the art.

Advantageously, a eukaryotic expression vector encoding the transgene may comprise a locus control region (LCR). LCRs are capable of directing high-level integration site independent expression of transgenes integrated into host cell chromatin, which is of importance especially where the transgene is to be expressed in the context of a permanently-transfected eukaryotic cell in which chromosomal integration of the vector has occurred, in vectors designed for gene therapy applications or in transgenic animals.

Vectors may be designed for precise integration into defined loci of the host genome, thus avoiding the disadvantages of random integration. Alternatively, artificial mammalian chromosomes may be used to deliver the genes of interest, thus avoiding any integration-related issues.

Eukaryotic expression vectors will also contain sequences necessary for the termination of transcription and for stabilizing the mRNA. Such sequences are commonly available from the 5′ and 3′ untranslated regions of eukaryotic or viral DNAs or cDNAs. These regions may contain nucleotide segments which direct polyadenylation of the messenger RNA during post-transcriptional processing thereof.

An expression vector includes any vector capable of expressing nucleic acids that are operatively linked with regulatory sequences, such as promoter regions, that are capable of expression of such DNAs. Thus, an expression vector refers to a recombinant DNA or RNA construct that, upon introduction into an appropriate host cell, results in expression of the cloned DNA. Appropriate expression vectors are well known to those with ordinary skill in the art and include those that are replicable in eukaryotic and/or prokaryotic cells and those that remain episomal or those which integrate into the host cell genome. For example, nucleic acids encoding a transgene may be inserted into a vector suitable for expression of cDNAs in mammalian cells, e.g. a CMV enhancer-based vector such as pEVRF (Matthias et al., (1989) NAR 17:6418).

The promoter and enhancer of the transgene are preferably strongly active, or capable of being strongly induced, in the primary target cells under conditions for production of the transgene product of interest. The promoter and/or enhancer may be constitutively efficient, or may be tissue or temporally restricted in their activity. Examples of suitable tissue restricted promoters/enhancers are those which are highly active in lymphoid cells, such as a promoter/enhancer from an antibody gene or a TCR gene. Examples of temporally restricted promoters/enhancers are those which are responsive to ischemia and/or hypoxia, such as hypoxia response elements or the promoter/enhancer of a grp78 or a grp94 gene. One preferred promoter-enhancer combination is a human cytomegalovirus (hCMV) major immediate early (MW) promoter/enhancer combination.

In one preferred aspect of the present invention, the transgene is hypoxia or ischemia regulatable. In this regard, hypoxia is a powerful regulator of gene expression in a wide range of different cell types and acts by the induction of the activity of hypoxia-inducible transcription factors such as hypoxia inducible factor-1 (HIP-1; Wang and Semenza, (1993) PNAS 90:430), which bind to cognate DNA recognition sites, the hypoxia-responsive elements (HREs) on various gene promoters. Dachs et al., (1997) Nature Med. 5:515) have used a multimeric form of the HRE from the mouse phosphoglycerate kinase-1 (PGK-1) gene (Firth et al., (1994) Proc. Natl. Acad. Sci. 91:6496-6500) to control expression of both marker and therapeutic genes by human fibrosarcoma cells in response to hypoxia in vitro and within solid tumors in vivo (Dachs et al., supra). Alternatively, the fact that marked glucose deprivation is also present in ischemic areas of tumors can be used to activate heterologous gene expression specifically in tumors. For example, a truncated 632 base pair sequence of the grp78 gene promoter, known to be activated specifically by glucose deprivation, has also been shown to be capable of driving high level expression of a reporter gene in murine tumors in vivo (Gazit et al., (1995) Cancer Res. 55:1660).

An alternative method of regulating the expression of a transgene is by using the tetracycline on/off system described by Gossen and Bujard ((1992) Proc. Natl. Acad. Sci. 89:5547) as described for the production of retroviral gal, pol and VSV-G proteins by Yoshida et al. ((1997) Biochem. Biophys. Res. Comm. 230:426). Unusually this regulatory system is also used in the present invention to control the production of the pro-vector genome, described further below. This ensures that no vector components are expressed from the adenoviral vector in the absence of tetracycline.

Construction of vectors according to the invention employs conventional ligation techniques. Isolated plasmids or DNA fragments are cleaved, and/or modified (e.g., phosphorylated, or kinased), and religated in a form desired to generate the plasmids required. If desired, analysis to confirm correct sequences in the constructed plasmids is performed in a known fashion (e.g., restriction enzyme digestion, sequencing, mini-sequencing, PCR, or combinations thereof). Suitable methods for constructing expression vectors, preparing in vitro transcripts (e.g., cDNAs), introducing DNA into host cells, and performing analyses for assessing expression and function are known to those skilled in the art. Gene presence, amplification and/or expression may be measured in a sample directly, for example, by conventional Southern blotting, Northern blotting to quantitate the transcription of mRNA, dot blotting (DNA or RNA analysis), or in situ hybridization, using an appropriately labeled probe.

Suitable techniques are fully described in the literature. See for example; Sambrook et al. (1989), In Molecular Cloning; A Laboratory Manual; Flames and Glover, (1985-1997) In DNA Cloning: A Practical Approach, Volumes I-IV (second edition). Methods for the engineering of immunoglobulin genes are given in McCafferty et al., (1996) In Antibody Engineering: A Practical Approach.

Those skilled in the art will readily envisage how these methods may be modified, if desired.

Viral vector systems include but are not limited to adenovirus vectors, adeno-associated viral (AAV) vectors, herpes viral vectors, retroviral vectors, lentiviral vectors and baculoviral vectors.

Viral vectors according to the present invention are preferably retroviral vectors. The term “retroviral vector” typically includes a retroviral nucleic acid which is capable of infection, but which is not capable, by itself, of replication. Thus it is replication defective. A retroviral vector typically comprises one or more transgene(s), preferably of non-retroviral origin, for delivery to target cells. A retroviral vector may also comprises a functional splice donor site (FSDS) and a functional splice acceptor site (FSAS) so that when the FSDS is upstream of the FSAS, any intervening sequence(s) are capable of being spliced. A retroviral vector may comprise further non-retroviral sequences, such as non-retroviral control sequences in the U3 region which may influence expression of an transgene(s) once the retroviral vector is integrated as a provirus into a target cell. The retroviral vector need not contain elements from only a single retrovirus. Thus, in accordance with the present invention, it is possible to have elements derivable from two of more different retroviruses or other sources

The term “retroviral pro-vector” typically includes a retroviral vector genome as described above but which comprises a first nucleotide sequence (NS) capable of yielding a functional splice donor site (FSDs) and a second NS capable of yielding a functional splice acceptor site (FSAS) such that the first NS is downstream of the second NS so that splicing associated with the first NS and the second NS cannot occur. Upon reverse transcription of the retroviral pro-vector, a retroviral vector is formed.

The term “retroviral vector particle” refers to the packaged retroviral vector, that is preferably capable of binding to and entering target cells. The components of the particle, as already discussed for the vector, may be modified with respect to the wild type retrovirus. For example, the Env proteins in the proteinaceous coat of the particle may be genetically modified in order to alter their targeting specificity or achieve some other desired function.

The retroviral vector of this aspect of the invention may be derivable from a murine oncoretrovirus such as MMLV, MSV or MMTV; or may be derivable from a lentivirus such as HIV-1 or EIAV; or may be derivable from another retrovirus.

The retroviral vector of the invention can be modified to render the natural splice donor site of the retrovirus non-functional.

The term “modification” includes the silencing or removal of the natural splice donor. Vectors, such as MLV based vectors, which have the splice donor site removed are known in the art. An example of such a vector is pBABE (Morgenstern et al. (1990) supra).

### Transgene Construction

In accordance with the present invention, the transgene can be any suitable nucleotide sequence. For example, the sequence may be DNA or RNA—which may be synthetically prepared or may be prepared by use of recombinant DNA techniques or may be isolated from natural sources or may be combinations thereof. The sequence may be a sense sequence or an antisense sequence. There may be a plurality of sequences, which may be directly or indirectly joined to each other, or combinations thereof.

A plurality of transgenes may be cloned as a tandem repeat, for example two or three copies of the transgene may be cloned as a tandem repeat, or even more.

Suitable transgene coding sequences include those that are of therapeutic and/or diagnostic application such as, but are not limited to: sequences encoding cytokines, chemokines, hormones, antibodies, engineered immunoglobulin-like molecules, a single chain antibody, fusion proteins, enzymes, immune co-stimulatory molecules, immunomodulatory molecules, anti-sense RNA, a transdominant negative mutant of a target protein, a toxin, a conditional toxin, an antigen, a tumor suppressor protein and growth factors, membrane proteins, vasoactive proteins and peptides, anti-viral proteins and ribozymes, and derivatives thereof (such as with an associated reporter group). When included, such coding sequences may be typically operatively linked to a suitable promoter, which may be a promoter driving expression of a ribozyme(s), or a different promoter or promoters.

The transgene may encode a fusion protein or a segment of a coding sequence. In a preferred aspect, the transgene is a “therapeutic gene” encoding a transgene product useful in a therapy (e.g., as the active ingredient of a pharmaceutical composition) and the regulatable population of cells comprising the transgene is used to produce amounts of transgene product suitable for therapies.

The delivery of one or more therapeutic genes according to the present invention may be used alone or in combination with other treatments or components of the treatment.

### Providing A Regulatable Population of Cells

The method comprises the steps of providing a host organism comprising a regulatable population of cells which express a transgene encoding the transgene product and administering an agent that regulates the number of transformed cells, thereby permitting the level of the transgene to be modulated. In one aspect, the regulatable population of cells is transformed with the transgene and introduced into the host organism (e.g., by intravenous injection, transplantation, delivery through a medical access device, such as a catheter and the like).

Regulatable cells according to the invention include cells whose proliferation and/or death can be selectively controlled upon administration of an agent. Exemplary cells include, but are not limited to, apoptotic cells, hematopoietic stem cells, antigen presenting cells (e.g, macrophages, monocytes, dendritic cell, a macrophage, a B cell, a mast cell, a parenchymal cell, a kupffer cell, or a fibroblast cell.), T cells, monocytes, basophils, natural killer cells, erthrocytes, megakaryocytes, platelets, basophils, endothelial cells, leukocytes, fibroblasts, endothelial cells, chondrocytes, osteoblasts, and their precusor cells. fibroblasts, eosinophils, keratinocytes, astrocytes, microglial cells, thymic cortical epithelial cells, Schwann cells, retinal pigment epithelial cells, myoblasts, vascular smooth muscle cells, enterocytes, thyrocytes and kidney tubule cells. Cells may be of epithelial, connective, muscle tissue, nervous tissue, so long as these are capable of modulating their numbers in response to an agent.

Regulatable cells preferably are obtained from a population of cells of substantially the same cell type. Preferably, the population of cells comprises 95% to 100% of cells of the same type, i.e., the cells comprise substantially the same genomic sequences (99%-100%) identity in the population and express substantially the same transcripts and polypeptides (95%, and preferably 99%-100% of transcripts, polypeptides in any given cell are the same as the transcripts/polypeptides in a cell which is “substantially the same”). A population of cells from an organism can be obtained by enriching for cells comprising characteristic cell surface markers such as described further below.

In one aspect, of the invention, cell populations according to the present invention are preferably blood cells, and advantageously lymphocytes. In general, such cells may be readily distinguished by the presence of surface markers known in the art (e.g., such as CD antigens), and thus may be isolated from tissues such as spleen or blood by any number of means known in the art such as one or more of: flow cytometry; magnetic sorting (e.g., using magnetic or superparamagnetic microbeads or particles coupled to antibodies which bind to surface markers); differential gradient centrifugation; dissection; panning using antibody-coated culture plates or containers; filtration; and the like.

For example, B lymphocytes, and especially memory B lymphocytes which are advantageously employed in the context of the present invention, may be obtained and isolated by immunization of an organism with a desired antigen, and selection of antigen-binding cells which express the B cell marker B220 and low levels of surface immunoglobulin differing in class from 1 gM and IgD as expressed by naive B cells, as set forth in Schittek and Rajewsky, (1990) Nature 346:749. In a preferred aspect, memory B cells may be identified by the presence of surface IgG. Similar methods may be applied to the isolation of T-lymphocytes. Preferably, the T cells also are antigen-specific (e.g., regulatable by exposure to antigen). Cloning of antigen-specific T cells is described in Simpson and Chandler, “Classical Techniques for Cellular Immunology”, in Weir's Handbook of Experimental Immunology, fifth edition, Eds. Herzenberg et al., (1997) Blackwell Science, Chapter 139, pp 139.9-1G.

Preferably, allogenic or syngeneic cells are used, although autologous cells and may also be used. In these embodiments, the host may be exposed to one or more immunosuppressants (e.g., such as methotrexate, cyclosporin, and the like) as are known in that art.

### Introducing Cells and Administering an Agent

Cell populations for use according to the invention may be transformed by any appropriate technique suitable for introduction of nucleic acids into cells, such as viral transduction, electroporation, transfection, or protoplast fusion. Protoplast fusion may be used by culturing bacterial cells, such as E. coli cells, comprising an episomal vector containing the transgene in a suitable culture broth. The vector may optionally be amplified in the bacterial cells before lysis of the cell walls to release bacterial protoplasts. Protoplasts are then combined with the cells according to the invention (e.g., such as mammalian cells) and mixed with a fusion-inducing agent, such as polyethylene glycol fusion reagent (PEG-FR). Alternatively, viral transduction methods may be used; Corcoran et al., (1996) EMBO 15:1924; Bonini et al., (1997) Science 276:5319, provide examples of virus-mediated gene transduction in T and B cells. Yoakum, et al., (1983) Science 222:385 and Fisher et al., (1985) Nature 31:262, provide references and a detailed protocol for protoplast fusion of lymphocytes. The regulatable population of cells can then be introduced into a host organism using methods known in the art (e.g., intravenous injection, transplantation, and the like).

Preferably, the host organism is member of the subphylum Chordata. Still more preferably, the host organism is a mammal, including, but not limited to humans and other primates, rodents (e.g., mice, rats, and guinea pigs), lagamorphs (e.g., rabbits), bovines (e.g, cattle), ovines (e.g., sheep), caprines (e.g., goats), porcines (e.g., swine), equines (e.g., horses), canines (e.g., dogs), felines (e.g., cats), domestic fowl (e.g., chickens, turkeys, ducks, geese, other gallinaceous birds, etc.), as well as feral or wild animals, including, but not limited to, such animals as ungulates (e.g., deer), bear, fish, lagamorphs, rodents, birds, etc. It is not intended that the term be limited to a particular age or sex. Thus, adult and newborn subjects, as well as fetuses, whether male or female, are encompassed by the term. In a particularly preferred embodiment, the host organism comprises an immune system.

This invention takes advantage of the many cells that can survive long-term in organisms and be induced to proliferate by a variety of means. For instance, it is known that memory B and T lymphocytes can persist, for seemingly indefinite periods, in immunologically compatible adoptive hosts. Moreover, it is known that B and T lymphocytes may be induced to proliferate, in homologous and adoptive hosts, by administration of antigen. See, for example, Sprent et al., (1991) J. Exp. Med. 174:717; Gray and Skarvall, (1988) Nature 336:70; Schittek and Rajewsky, (1991) Nature 346:749; Ahmed and Gray, (1996) Science 272:54; Bruno et al., (1995) Immunity 2:37; Farber, (1998) J. Immunol. 160:535; Markiewicz et al., (1998) PNAS (USA) 95:3065; and Sprent, (1997) Curr. Opin. Immunol. 9:371.

Accordingly, in one aspect, the invention takes advantage of the clonal expansion of lymphocytes in response to antigen presentation, providing the host organism with a regulatable population of cells which comprise lymphocytes transformed with a transgene of interest and administering to the host organism an agent which comprises an antigen or comprises a nucleic acid which encodes an antigen.

Routes and frequency of antigen administration, as well as dosage may be varied. In one aspect, antigens are administered by injection (e.g., intracutaneous, intramuscular, intravenous or subcutaneous), intranasally (e.g., by aspiration) or orally. A primary immunization may be followed by one or more booster immunizations. For example, 1, 2, 3 or more doses may be administered for a 1-36 week period. Preferably, 3 doses are administered, at intervals of 3-4 months. A suitable dose is an amount of antigen that, when administered as described above, is capable of raising an immune response in an immunized host organism sufficient to increase or decrease the numbers of cells in the regulatable population of cells by at least 1%, at least 5%, at least 10%, 20%, 30%, 40%-100%, or at least 2-fold, 4-fold, 10-fold, 20-fold, 50-fold, etc. In general, the amount of antigen in a dose (or produced in situ by DNA encoding antigen in a dose) ranges from about 1 pg to about 100 mg per kg of host, typically from about 10 pg to about 1 mg, and preferably from about 100 pg to about 1 μg. Suitable dose sizes will vary with the size of the host organism, but will typically range from about 0.1 mL to about 5 mL.

While any suitable carrier may be employed to facilitate administration of the antigen, the type of carrier will vary depending on the mode of administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactic galactide) may also be employed as carriers. Suitable biodegradable microspheres are disclosed, for example, in U.S. Pat. Nos. 4,897,268 and 5,075,109.

Any of a variety of adjuvants may be employed to nonspecifically enhance the immune response. Most adjuvants contain a substance designed to protect the antigen from rapid catabolism, such as aluminum hydroxide or mineral oil, and a nonspecific stimulator of immune responses, such as lipid A, Bortadella pertussis or Mycobacterium tuberculosis. Suitable adjuvants are commercially available as, for example, Freund's Incomplete Adjuvant and Freund's Complete Adjuvant (Difco Laboratories) and Merck Adjuvant 65 (Merck and Company, Inc., Rahway, N.J.). Other suitable adjuvants include alum, biodegradable microspheres, monophosphoryl lipid A and quil A.

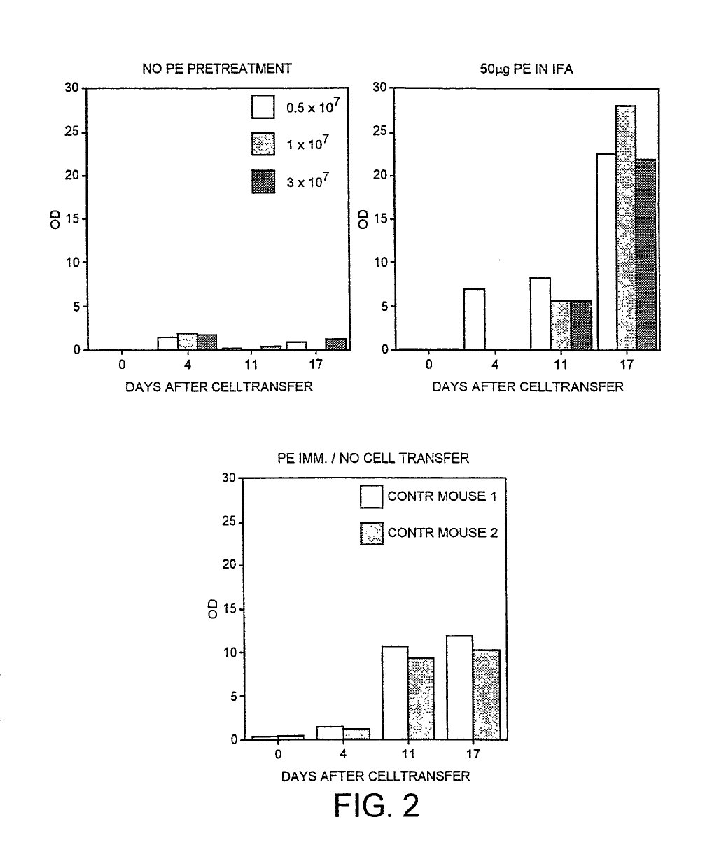

Examples of protein antigens and derivative peptides used successfully to elicit antigen-specific T cell responses are given in Harlow and Lane, (1988) In Antibodies: A Laboratory Manual, Cold Spring Harbor, N.Y., USA; chapter 5, page 132, FIG. 2, reproduced in part below as Table 1.

TABLE 1
Immunogenic Proteins And Peptides Useful For Generating Antigen Specific T Cells

| Protein* | Residues | Sequence | Reference |
|---|---|---|---|
| Sperm Whale | 68-78 | VLTALGAILKK | Livingstone and Fathman (1987) |
| Myoglobin | 102-118 | KYLEFISEABIIVLHSR | Cease et al. (1986) |
| | 132-146 | NKALELFRKDIAAKY | Berkower et al. (1986) |
| Chicken | 46-61 | NTDGSTDYGILQINSR | Allen et al. (1985) |
| Lysozyme | 74-86 | NLCNJPCSALLSS | Shastri et al. (1985) |
| | 81-96 | SALLSSDITASVNCAK | Shastri et al. (1985) |
| | 108-119 | WVAWRNRCKGRD | Katz et al. (1982) |
| Chicken | 323-339 | ISQQVHAAHAENEAGR | Shimonkevitz et al. (1984) |
| Ovalbumin | | | |
| Pig | 94-104 | LIAYLKQATAK | Schwartz et al. (1985) |
| Cytochrome c | | | |
| Influenza HA | 109-119 | SSFERFEIFPK | Hackett et al. (1983) |
| from A/PR8/48 | 129-140 | NGVTAACSHEGK | Hurwitz et al. (1984) |
| | 302-313 | CPKYVRSAKLRM | Hurwitz et at (1984) |
| Hepatitis B | 38-52 | SLNFLGGITVCLGQN | Milich et al. (1985) |
| S Antigen | 95-109 | LVLLDYQGMLPVCPL | Milich et al. (1985) |
| | 140-154 | TK PE DGNCTCIPITS | Milich et al. (1985) |
| Lambda | 12-26 | QLEDARRLKAIYEKK | Guillet et al. (1986) |
| Repressor | | | |

The induction of an antigen-specific immune response may be monitored using methods routine in the art such as assays for, e.g., antibody production, lymphocyte proliferation, cell-mediated cytotoxicity, or cytokine production which is significantly higher, in an organism treated with the antigen vs. a control organism which is not exposed to the antigen. Preferably, the control organism also comprises a transformed population of regulatable cells. In general, bodily fluids of the host organism may be monitored to determine the relative numbers of a circulating regulatable cell. The relative numbers of other types of regulatable cells can be evaluated by imaging agents selective for those types of cells (e.g., such as labeled antibodies reactive with the cells) and can be monitored, for example, by imaging techniques, such as MRI, comparing the number of labeled cells in host organisms exposed to an agent vs. host organsims not exposed to the agent.

In one aspect, modulation in the numbers of regulatable cells is monitored indirectly, through monitoring the levels of the modulatable transgene product as discussed further below. Preferably, the induction of antigen-specific cell regulation results in modulation of the levels of the transgene product encoded by the transgene by virtue of changes in the number of regulatable cells in the host organism which comprise the transgene product (although additional levels of transcriptional and/or translational control may also be provided as discussed above). In one aspect, modulating means an increase or decrease of at least 1% of the total transgene product produced. In another aspect, the increase or decrease in the total transgene product produced is at least 5%-25%, or at least 25% to 50%. In addition, the change in the level of transgene product may be an at least 2-, 4-, 5-, 10-, 20-, 25- or 50-fold increase or decrease in the amount of transgene product in the host organism after exposure to the agent when compared to a host organism comprising the transformed regulatable population of cells not exposed to the antigen/agent.

As discussed above, agents in addition to antigens can be used to regulate the numbers of a regulatable population of cells. For example, selected cytokines induce lymphocyte (F-cell or B cell) proliferation, or proliferation of other blood cells such as macrophages, neutrophils or other leukocytes, mast cells and antigen-presenting cells. Growth factors also can be used as agents. Agents which induce proliferation also can be used in combination with agents which suppress apoptosis (e.g., bcl-2, SB343 1, c-IAP-2, and the like). In addition, agents such as bclxs, myc, p53, bax, bak, ced transgene products, Fas ligand (FasL), Fas receptor, lymphotoxin, CD40L, and TNF- alpha can be used to reduce cell numbers, e.g., by inducing apoptosis.

Growth factors or hormones which act on any of the above regulatable cell types are encompassed within the scope of the invention, including, but not limited to: Stem Cell Factor (SCF; also known as mast cell growth factor, or Steel Factor), TGF-alpha, TGF-beta, VEGF, macrophage colony stimulating factor (M-CSF), keratinocyte growth factor (KGF), heregulin (HRG), insulin, insulin-like growth factors I and II (IGF-I and IGF-II), epidermal growth factor (EGF), interleukins (e.g., IL-8), macrophage colony-stimulating factor (M-CSF), erythropoietin (EPO), platelet-derived growth factor (PDGF), fibroblast growth factor (FGF), transforming growth factors alpha and beta (TGF- [alpha] and TGF- [beta]), hepatocyte growth factor (HGF), and nerve growth factor (NGF).

Agents are not limited to polypeptides and may be chemical substances or biological organisms, such as an infecting organism or a virus that has been optionally engineered to express a molecule/agent that modifies cell number or a virus/infecting organism which itself may be cytolytic (e.g., a paramyxovirus) or transforming (e.g., such as an oncogenic virus such as EBV).

In a preferred aspect of the invention, the expression of the modulatable transgene product is monitored by sampling a bodily fluid of the host organism, for example, by obtaining blood, or by sampling a tissue (e.g., such as skin). The transgene product may be detected using methods routine in the art for assaying nucleic acids or polypeptides or other agents. For example, in the case of a nucleic acid transgene product, a hybridization based assay (e.g., Southern, Northern, amplification assay) may be used, while in the case of a polypeptide transgene product, immunoassays may be employed. Immunoassays include, but are not limited to, radioimmunoassays, enzyme immunoassays (e.g. ELISA), immunofluorescence (such as immunohistochemical analyses), immunoprecipitation, latex agglutination, hemagglutination, and histochemical tests. Chromatographic methods such as HPLC, optionally combined with mass spectrometry, also can be employed. Such assays are routine in the art.

### Transgenic Animals Comprising Regulatable Cells

In a different aspect of the invention, the regulatable population of cells is obtained from the progeny of a transformed progenitor population of cells, e.g., such as from animals whose germline has been transformed with the transgene. Therefore, the invention also provides for transgenic animals that include but are not limited to: transgenic mice, rabbits, rats, pigs, sheep, horses, cows, goats, non-human primates, etc. A protocol for the production of a transgenic pig can be found in White and Yannoutsos, Current Topics in Complement Research: 64^{th} Forum in Immunology, pp. 88-94; U.S. Pat. No. 5,523,226; U.S. Pat. No. 5,573,933: PCT Application WO93/25071; and PCT Application WO95/04744. A protocol for the production of a transgenic mouse can be found in U.S. Pat. No. 5,530,177. A protocol for the production of a transgenic rat can be found in Bader and Ganten, (1996) Clinical and Experimental Pharmacology and Physiology, Supp. 3:S81-S87. A protocol for the production of a transgenic goat may be found in Ko et al., (2000) Transgenic Res. 9(3):215-22. A protocol for the production of a transgenic cow can be found in Transgenic Animal Technology, A Handbook, 1994, ed., Carl A. Pinkert, Academic Press, Inc. A protocol for the production of transgenic sheep can be found in Mezina et al., (2001) Biochemistry (Mosc). 66(4):378-83. A protocol for the production of a transgenic rabbit can be found in Hammer et al., (1985) Nature 315:680-683 and Taylor and Fan, (1997) Frontiers in Bioscience 2:d298-308. A protocol for the production of a transgenic monkey may be found in Wolfgang et al., (2001) J. Med. Primatol. 30(3):148-55.

The transgenic host organisms according to the invention can be produced by introducing transgenes into the germline of the animal, particularly into cells which can proliferate to generate bone marrow cells, such as hematopoietic cells. Embryonic target cells at various developmental stages can be used to introduce the transgene constuct. As is generally understood in the art, different methods are used to introduce the transgene depending on the stage of development of the embryonal target cell. Appropriate targets for germline gene transfer include oocytes, stem cells, and the like.

In one technique for producing transgenic animals, fertilized oocytes are first taken from female animals. Desired foreign DNA encoding a modulatable transgene product (the “transgene”) is then incorporated into the genome of the oocytes. For example, the transgene can be incorporated via an appropriate retroviral vector or by microinjection (see, e.g., as described in Palmiter and Brinster, (1986) Ann. Rev. Genet. 20:465-499). Recombinant retroviral vectors are incorporated into the oocytes according to processes known from the prior art (see, e.g., Jaenisch, (1988) Science 240:1468-1475). In the microinjection, exogenous DNA is injected directly into one of the two pronuclei of a fertilized oocyte prior to the fusion of the pronuclei of sperm and oocytes. The oocytes are then reimplanted into pseudopregnant females for gestation.

Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic; where it is transgenic, it may contain the same or a different transgene, or both. Where in vitro fertilization is used, the fertilized embryo may be implanted into a surrogate host or incubated in vitro, or both. Progeny may be evaluated for the presence of the transgene using methods routine in the art. Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic and/or a knockout; where it is transgenic, it may contain the same or a different transgene, or both. Where in vitro fertilization is used, the fertilized embryo may be implanted into a surrogate host or incubated in vitro, or both. Progeny may be evaluated for the presence of the transgene using methods routine in the art. Both heterozygous and homozygous animals are encompassed within the scope of the invention as are chimeric animals.

In the case of embryonal stem cell (ES), the cells are obtained from pre-implantation embryos cultured in vitro and fused with embryos (Evans et al., (1981) Nature 292:154-156; Bradley et al., (1984) Nature 309:255-258; Gossler et al., (1986) PNAS 83:9065-9069; and Robertson et al., (1986) Nature 322:445-448). Any line of ES cells can be used; however, the line chosen is typically selected for the ability of the cells to integrate into and become part of the germ line of a developing embryo so as to create germ line transmission of the knockout construct. Non-mouse embryonic stems are known and also are encompassed within the scope of the invention (see, e.g., Talbot et al., (2001) Anat. Rec. 264(1):101-13 (pig); Chen et al., (1999), Theriogenology 52(2): 195-212 (pig); Iwazaki et al., (2000) Biol. Reprod. 62(2):470-5 (cow); Schoonjans et al., (1996) Mol Reprod Dev. 45(4): 439-43 (rabbit); Li et al., (2001) Blood 98(2): 335-42 (monkey); Marshall et al., (2001) Methods Mol. Biol. 158: 11-8 (monkey)).

Insertion of a transgene into ES cells can be accomplished using a variety of methods well known in the art including, for example, electroporation, microinjection, and calcium phosphate treatment. A preferred method of insertion is electroporation. More than one type of construct can be introduced into an ES cell, simultaneously or sequentially. Transgenes also can be efficiently introduced retrovirus-mediated transduction. Such transformed ES cells can thereafter be combined with blastocysts from the same species. The ES cells can be used thereafter to colonize the embryo and contribute to the germline of the resulting chimeric animal which can be bred to siblings or other chimeric animals to obtain homozygotes or heterozygotes comprising populations of cells comprising the transgene. For review, see Jaenisch, 1988, supra.

Screening for transgenic animals comprising transgene nucleic acids can be accomplished by a number of ways. For example, Southern blots of genomic DNA can be probed with a sequence of DNA designed to hybridize only to transgenic sequences. Alternatively, PCR can be used. Northern blots also can be used to probe mRNA of candidate transgenic animals for the presence or absence of transcripts encoding either the transgene. In addition, immunoassays can be used to assess the level of expression of polypeptides encoded by the transgene in tissues of the host organism or in bodily fluids from the host organism. Selectable marker genes also can be provided as part of a vector comprising the transgene and the presence or absence of the marker transgene product can be detected.

Introduction of the recombinant gene at the fertilized oocyte stage ensures that the gene sequence will be present in all of the germ cells and somatic cells of the transgenic “founder” animal. The presence of the transgene in the germ cells of the transgenic founder animal in turn means that approximately half of the founder animal's descendants will carry the activated recombinant gene sequence in all of their germ cells and somatic cells. Introduction of the transgene at a later embryonic stage might result in the gene's absence from some somatic cells of the founder animal, but the descendants of such an animal that inherit the gene will carry the activated recombinant gene in all of their germ cells and somatic cells.

Tissue-specific control of a transgene's expression (e.g., in a regulatable population of cells) can be controlled by the selection of appropriate promoters as described above. In one aspect, tissue specific control is achieved using a recombinase/recombinase recognition sequence system to generate transgenic animals. For example, the cre/loxP recombinase system of bacteriophage P1 (Lakso et al., (1992) PNAS 89:6232-6236; Orban et al., (1992) PNAS 89:6861-6865). Cre recombinase catalyzes the site-specific recombination of an intervening target sequence located between loxP sequences. LoxP sequences are 34 base pair nucleotide repeat sequences to which the Cre recombinase binds and are required for Cre recombinase-mediated genetic recombination. The orientation of loxP sequences determines whether the intervening target sequence is excised or inverted when Cre recombinase is present (Abremski et al., (1984) J Biol. Chem. 259:1509-1514). When loxP sequences are oriented as direct repeats intervening sequences are excised upon binding of Cre. When loxP sequences are oriented as inverted repeats, intervening sequences are inverted. Expression of the recombinase can be regulated by promoter elements which are subject to regulatory control, e.g., tissue-specific promoters as are known in the art. This regulated control will result in genetic recombination of the target sequence only in cells where Cre recombinase expression is mediated by the promoter element. Thus, expression of a modulatable transgene product produced by a transgene can be regulated in a tissue-specific manner via control of Cre recombinase expression. The FLP recombinase system of Saccharomyces cerevisiae (O'Gorman et al., (1991) Science 251:1351-1355; WO 92/15694) can be used in a similar manner to mediate tissue-specific regulation of transgene products.

In one aspect of the invention, where the genome of all cells of the host organism comprise the transgene, expression of the transgene in the regulatable population of cells can be achieved as described above using appropriate promoters. Administration of the agent to the transgenic host organism will modulate the number of the regulatable cells in the host organism thereby increasing or decreasing the level of transgene product depending on the nature of the agent as described above. However, in another aspect, regulatable cells can be obtained from the transgenic host animal, and substantially enriched for (e.g., providing a population of cells comprising greater than 80%, preferably, greater than 90%, and more preferably greater than 95% of the regulatable cells) and these can be introduced into a second host organism (transgenic or non-transgenic) using methods known in the art. The agent can be administered to the second host organism to modulate the number of regulatable cells in the second host organism as described above.

### Methods of Using the Modulated Transgene Products

In one aspect, modulating the number of regulatable cells comprising the transgene expressing the transgene product, enables isolation and purification of amounts of transgene product which are suitable for preparing pharmaceutical compositions whose active ingredients comprise the transgene product. Preferably, miligram to gram quantities of transgene product are produced using host organisms according to the invention.

Such pharmaceutical compositions can be used in the treatment of the disorders listed in WO-A-98/05635. For ease of reference, part of that list is now provided: cancer, inflammation or inflammatory disease, dermatological disorders, fever, cardiovascular effects, hemorrhage, coagulation and acute phase response, cachexia, anorexia, acute infection, HIV infection, shock states, graft-versus-host reactions, autoimmune disease, reperfusion injury, meningitis, migraine and aspirin-dependent anti-thrombosis; tumor growth, invasion and spread, angiogenesis, metastases, malignant, ascites and malignant pleural effusion; cerebral ischemia, ischemic heart disease, osteoarthritis, rheumatoid arthritis, osteoporosis, asthma, multiple sclerosis, neurodegeneration, Alzheimer's disease, atherosclerosis, stroke, vasculitis, Crohn's disease and ulcerative colitis; periodontitis, gingivitis; psoriasis, atopic dermatitis, chronic ulcers, epidermolysis bullosa; conical ulceration, retinopathy and surgical wound healing; rhinitis, allergic conjunctivitis, eczema, anaphylaxis; restenosis, congestive heart failure, endometriosis, atherosclerosis or endosclerosis.

In addition, or in the alternative, the method of the present invention may be used to deliver one or more transgene products useful in the treatment of disorders listed in WO-A-98/07859. For ease of reference, part of that list is now provided: cytokine and cell proliferation/differentiation activity; immunosuppressant or immunostimulant activity (e.g. for treating immune deficiency, including infection with human immune deficiency virus; regulation of lymphocyte growth; treating cancer and many autoimmune diseases, and to prevent transplant rejection or induce tumor immunity); regulation of hematopoiesis, e.g. treatment of myeloid or lymphoid diseases; promoting growth of bone, cartilage, tendon, ligament and nerve tissue, e.g. for healing wounds, treatment of burns, ulcers and periodontal disease and neurodegeneration; inhibition or activation of follicle-stimulating hormone (modulation of fertility); chemotactic/chemokinetic activity (e.g. for mobilizing specific cell types to sites of injury or infection); hemostatic and thrombolytic activity (e.g. for treating hemophilia and stroke); anti-inflammatory activity (for treating e.g. septic shock or Crohn's disease); as antimicrobials; modulators of e.g. metabolism or behavior; as analgesics; treating specific deficiency disorders; in treatment of e.g. PE oriasis, in human or veterinary medicine.

In addition, or in the alternative, the method of the present invention may be used to deliver one or more transgene products useful in the treatment of disorders listed in WO-A-98/09985. For ease of reference, part of that list is now provided: macrophage inhibitory and/or T cell inhibitory activity and thus, anti-inflammatory activity; anti-immune activity, i.e. inhibitory effects against a cellular and/or humoral immune response, including a response not associated with inflammation; inhibit the ability of macrophages and T cells to adhere to extracellular matrix components and flbronectin, as well as upregulated fas receptor expression in T cells; inhibit unwanted immune reaction and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimoorchitis, infertility, orchidal trauma or other immune-related testicular diseases, placental dysfunction, placental insufficiency, habitual abortion, eclampsia, pre-eclampsia and other immune and/or inflammatory-related gynecological diseases, posterior uveitis, intermediate uveitis, anterior uveitis, conjunctivitis, chorioretinitis, uveoretinitis, optic neuritis, intraocular inflammation, e.g. retinitis or cystoid macular edema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumor cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

The dosage and administration of a therapeutically active amount of a composition may vary according to factors such as the disease state, age, sex, and weight of the patient, and dosage may be adjusted using methods routine in the art to provide the optimum therapeutic response. For example, several divided doses may be administered daily or the dose may be proportionally reduced or increased as indicated by monitoring one or more therapeutic endpoints. Dose size, number of doses, and mode of administration can be determined and optimized using methods known in the art (see, e.g., Hardman et al., (1995) Goodman and Gilman's In The Pharmacological Basis of Therapeutics, Ninth Edition, McGraw-Hill).

The pharmaceutical compositions according to the invention may be administered in a convenient manner such as by injection (subcutaneous, intravenous, etc.), oral administration, by inhalation, transdermal application, by vaginal or rectal administration. Depending on the route of administration, the active substance may be coated in a material to protect the compound from the action of enzymes, acids and other natural conditions which may inactivate the compound. Carriers and coatings are known in the art. Examples of such carriers include, but are not limited to water, phosphate buffered saline, saline, Ringer's solution, dextrose solution, serum-containing solutions, Hank's solution and other aqueous physiologically balanced solutions or cell culture medium.

Aqueous carriers can also contain suitable auxiliary substances required to approximate the physiological conditions of a recipient, for example, by enhancement of chemical stability and isotonicity. Suitable auxiliary substances include, for example, sodium acetate, sodium chloride, sodium lactate, potassium chloride, calcium chloride, sorbitan monolaurate, triethanolamine oleate, and other substances used to produce phosphate buffer, Tris buffer, and bicarbonate buffer. Auxiliary substances can also include preservatives, such as thimerosal, m or o-cresol, formalin and benzyl alcohol. Preferred auxiliary substances for aerosol delivery include surfactant substances nontoxic to a recipient, for example, esters or partial esters of fatty acids containing from about 6 to about 22 carbon atoms. Examples of esters include, caproic, octanoic, lauric, palmitic, stearic, linoleic, linolenic, olesteric, and oleic acids. Standard formulations can either be liquid injectables or solids which can be taken up in a suitable liquid as a suspension or solution for injection.

In another aspect of the invention, however, it is contemplated that transformed regulatable cells are administered to a host organism in need of a modulatable amount of a transgene product. For example, regulatable transformed cells can be provided to a human patient comprising any of the above-mentioned conditions as a means of providing a therapeutic agent whose levels can be controlled at least in part through administration of the agent. The regulatable transformed population of cells can be obtained from the patient, from a genetically related individual, or from a non-related individual (although in this scenario, it is contemplated that the patient will be treated with one or more immunosuppressive agents), and administered to the patient. In one aspect, the agent which stimulates the proliferation of the regulatable cells is administered at a level and amount of time necessary to achieve a therapeutic effect.

### EXAMPLES

The invention will now be further illustrated with reference to the following examples. It will be appreciated that what follows is by way of example only and that modifications to detail may be made while still falling within the scope of the invention.

### Example 1

### Generation of hF.IX Transgenic Mice

In order to assess clonal expansion of a population of cells expressing a therapeutic product in vivo, transgenic mice are prepared which express human Factor IX, as follows.

The Ig heavy chain locus enhancer Eμ (Neuberger et al., (1983) EMBO J. 2:1373) and placed 5′ to an Ig λI promoter (Bothwell et al., (1982) Nature 298:380). A human Factor IX coding sequence is constructed from genomic and cDNA sequences, as described in Kurachi et al., (1995) J. Biol. Chem. 270:5276 and placed downstream of the promoter/enhancer. A human β-globin 3′ terminator and polyadenylation signal is used, as described in Kemball-Cook et al., (1994) Gene 139:275.

A second construct, similar to the first, is made. This construct omits the Eμ enhancer, but additionally possesses an Igλ enhancer (Flagman et al., (1990) Genes and Dev. 4:978) 3′ to the Factor IX coding sequence.

The constructs are microinjected into murine oocyte pronuclei and used to generate transgenic mice as described in Houdebine, Transgenic animals—Generation and Use (Harwood Academic, 1997).

Tissue specificity of F.IX expression is determined by RT PCR, which shows high-level expression in spleen and thymus, and low or negligible expression in lung, liver, kidney and pancreas. Expression of FAX in B-lymphocytes is confirmed by preparation of pre-B cell lines therefrom, by Abelson virus transformation. These B cells express human F.IX.

The levels of F.IX expressed in the founder animals are shown in Table 2. FAX levels are determined by ELISA according to Walter et al., (1996) PNAS 93:3056, using as a first layer polyclonal rabbit anti-huF.IX (Dako), and as a second layer polyclonal goat anti-huF.IX coupled to horseradish peroxidase (Affinity Biologicals).

TABLE 2

| F.IX transgenic lines | hF.IX in founder sera | HF.IX in progeny sera |
|---|---|---|
| Eμ enhancer | (ng/ml) | (ng/ml) |
| 2 | 0 | |
| | 15 | 10-55 |
| 13 | 20 | |
| 21 | 115 | 200-500 |
| 28 | 315 | 650-1200 |
| 39 | <6 | |
| 45 | 300 | |
| 46 | 1200 | |
| 52 | 480 | |
| 53 | <6 | |
| 55 | 260 | |
| 59 | <6 | |
| Ig λ enhancer | | |
| 93 | 62 | |

### Example 2

### Adoptive Transfer of B Cells

Transgenic mice expressing 650 ng/ml hF.IX in serum are immunized with PE by intraperitoneal injection of 100 μg Alu-precipitated PE together with 10⁹ heat-killed B pertussis cells as an adjuvant. After two weeks, tail blood is analysed by ELISA and the anti-PB antibody Se determined to be 1:10,000.

Cells are prepared from the spleens of PE-immunized transgenic animals, and transferred to mice in the presence or absence of 50 μg PE in IFA (Incomplete Freunds Adjuvant). As a control, mice are also injected with 50 μg PE in WA in the absence of cell transfer. The number of cells transferred is 0.5×10¹, 1×10⁷ or 3×10⁷ for each mouse.

Tail blood is taken and analysed for anti-PB antibodies 4, 11 and 17 days after adoptive transfer. Booster immunizations with PE (50 μg in IFA) are given 13 and 26 days after transfer.

FIG. 2 shows the results obtained in mice immunized with PE alone (no cells), mice given cells with PE and mice given cells alone. A large increase of anti-PE antibody production, above control levels, can be seen mice receiving PE and PE-reactive spleen cells. This increase is indicative of increase of numbers of adoptively-transferred cells in the mice.

### Example 3

### Production of hEpo Transgenic Mice

Transgenic mice carrying the human erythropoietin (Epo) gene are prepared as described above in respect of hF.IX, using an Eμ enhancer and Igλ1 promoter. Epo production levels are assessed in sera of founder mice, and the results shown in Table 3. Epo levels are determined using a commercial Epo ELISA kit (EPO ELISA, Roche Diagnostics GmbH) according to the instructions provided by the manufacturer.

TABLE 3

| hEpo transgenic lines | hEpo in founder sera (mIU/ml) |
|---|---|
| 2 | 234 |
| 8 | 150 |
| 32 | |
| 52 | 354 |
| 54 | 294 |

Cells are derived from transgenic mouse spleens as described in Example 2, and used for adoptive transfer to PE-preimmunized and unimmunized mice, as in Example 2.

Boosting with PE leads to expansion of numbers of cells which produce Epo, and an increase in detectable Epo levels in mouse sera.

### Example 4

### Immunization of Mice

Mice are immunized with phycoerythrin (PE) polypeptide according to the methods set forth in Harlow and Lane, “Antibodies—A Laboratory Manual”, (1988) Cold Spring Harbor, N.Y., USA, chapter 5; immunizations).

For each mouse, 250 μl of PE solution (20 μg total) is mixed with 250 μl of complete Freunds adjuvant, and injected subcutaneously. The inoculation is repeated at day 14.

Tail blood is collected at day 24 and tested for PE-specific antibody response by titration on antigen-coated plates using ELISA.

At day 35, 250 μl of antigen (20 μg) with 250 μl of incomplete Freunds adjuvant is injected, to “boost” immunization according to levels of antibody response detected (see Harlow and Lane, chapter 5, page 114, and “adjuvants” chapter 5 page 96).

When antibody response is high and specific, a final boost is administered (100 μl (10 μg) of antigen solution given i.p. and 100 μl (10 μg) of antigen solution given i.v.) After a further 3 days spleen (or blood) mononuclear cells are harvested from the mice.

### Example 5

### In Vitro Stimulation and Transfection

B cells with receptors recognizing PE are purified from spleen or blood tissues by immunostaining and FACS sorting (PE is a fluorescent protein). These cultures are restimulated in vitro with Factor IX, or alternatively with anti-CD40 antibody (FGK.45, 10 μg/ml) and IL-4 to stimulate cell proliferation.

A transgene for expression of human Factor IX is constructed as follows: the murine λ light chain enhancer and LCR are coupled to the murine λ1 promoter, transcriptional start site and first intron, fused to human Factor IX cDNA, and cloned in pBluescript. A poly-A addition site is included downstream of the Factor DC cDNA. The transcription unit thus constructed is then cloned as a triple tandem repeat, always in pBluescript, to ensure efficient expression.

The transgene is introduced into cells (approx. 10⁶ target lymphocytes) by protoplast fusion, as follows: single colonies of bacteria bearing pBluescript containing the triple tandem repeat of the transgene are grown in L-broth containing ampicillin to a density of 2-4×10³ ml⁻¹ and chloramphenicol is added to a final concentration of 250 μg per ml of broth. After incubation at 37° C. for 2 hours, gentamycin is added to 10 μg ml⁻¹ and the cultures are re-incubated for 14-18 hours to allow plasmid amplification. The bacteria are collected and resuspended in 2.5 ml of HEPES-buffered saline (HBS) pH 8.0, containing 20% sucrose. This suspension is placed on ice and at 5-mm intervals, 0.8 ml of lysozyme solution (10 mg mi¹ in 0.25 M Tris-HCl pH 7.0), 1.0 ml of 0.25 M EDTA pH 8.0, and 1.0 ml of HBS are added sequentially. The suspension is incubated at 37° C. for 10-30 min until >90% of the bacteria have formed protoplasts, then held on ice and slowly diluted to 50 ml with RPMI-1640 medium. For each fusion 10¹⁰ bacterial protoplasts are combined with 1×10⁶ stimulated lymphocytes, washed and the pellet resuspended in 0.1 ml of RPM1-1640 medium. To these cells, 0.8 ml of polyethylene glycol fusion reagent (PEG-FR) is added dropwise over 1 min. The cells are left to stand for a further minute and then gradually diluted with RPMI-1640. After fusion cells are returned to culture in media supplemented with 100 μg/ml gentamycin antibiotic.

### Example 6

### Transfer to Host, Immunological Challenge and Monitoring

Transfection/infection of 10⁶ cells with the transgene construct results in stable transgene integration in between 100-1000 cells. These cells are introduced i.v. into recipient (allotype marked) mice and the effect of FE immunization (3 days, 10 days, 28 days and at subsequent dates), on levels of transgene protein in the serum, and anti-IgG (to PE protein) is monitored.

Human Factor IX expression is monitored by a sandwich ELISA technique using two Factor IX antibodies, as described by Alexander et al., (1995) Hum. Mol. Genet. 4:993-999, and Gerrard et al., (1993) Nature Genetics 3:180-183. This assay specifically detects human Factor IX against the murine background.

Human Factor IX expression is seen to be correlated with PE immunization, increasing after each immunization and remaining stable for an extended period of time after the final booster.

All of the references identified above, are hereby expressly incorporated herein by reference to the extent that they describe, set forth, provide a basis for or enable compositions and/or methods which may be important to the practice of one or more embodiments of the present inventions.

Variations, modifications, and other implementations of what is described herein will occur to those of ordinary skill in the art without departing from the spirit and scope of the invention as described and claimed herein.

What is claimed is:

1. A method for modulating the amount of a transgene product in a host organism, comprising comprising:
a) transforming a regulatable population of cells with a transgene encoding the gene product;
b) introducing the population of transformed cells into the host allowing for the transgene to be expressed and the transgene product to be produced in the host organism; and
c) administering an agent that regulates the number of the transformed cells, thereby modulating the amount of transgene product produced in the host organism.

2. The method according to claim 1, wherein step c) the number of transformed cells increases.

3. The method according to claim 1, wherein step c) the number of transformed cells decreases.

4. The method according to claim 1, wherein the regulatable population of cells is a clonal population of cells.

5. The method according to claim 1, wherein the regulatable population of cells comprises blood cells.

6. The method according to claim 5, wherein the blood cells comprise lymphocytes.

7. The method according to claim 1, wherein the host organism is a mammal.

8. The method according to claim 1, wherein the agent which regulates cell number is a cytokine.

9. The method according to claim 4, wherein the agent which regulates cell number is an antigen and the lymphocytes specifically proliferate in response to the antigen.

10. A method for modulating the amount of a transgene product in a host organism, comprising the steps of:
a) immunizing the host with the antigen in order to induce an immune response;
b) isolating lymphocytes from the host;
c) transforming the lymphocytes with a transgene encoding the transgene product;
d) reintroducing the lymphocytes to the host; and
e) administering a booster immunization of the antigen to the host in order to induce clonal expansion of the transformed lymphocytes.

11. The method according to claim 10, wherein step a) comprises a primary immunization and one or more booster immunizations.

12. The method according to claim 10, wherein step c) further comprises expanding the population of cells in vitro.

13. The method according to claim 10, wherein step b) further comprises enriching the isolated cells for lymphocytes.

14. The method according to claim 1, wherein the transgene product is a polypeptide.

15. The method according to claim 14, wherein the polypeptide is selected from the group consisting of an enzyme, a transcription factor, a growth factor, a hormone, a toxin, an antibody, a clotting factor, apolipoprotein Al, α-1 antitrypsin and a peptide drug.

16. The method according to claim 15, wherein the clotting factor is Factor VIII or Factor IX.

17. The method according to claims 1, wherein the transgene product of interest is a nucleic acid.

18. The method according to claim 1 or 10, wherein the transgene product is RNA.

19. The method according to claim 18, wherein the RNA is an antisense RNA or a ribozyme.

20. The method according to claim 1 or 10, wherein the transgene product is a viral polypeptide or viral transcript.

21. The method according to claim 20, wherein the virus polypeptide or viral transcript is encoded by a replication defective viral sequences.
